(19) United States
(12) Patent Application Publication
(10) Pub. No.: US 2006/0233858 A1
(43) Pub. Date: Oct. 19, 2006

(54) Systems and methods providing targeted intraocular drug delivery

(75) Inventors: Radouil T. Tzekov, Irvine, CA (US); Patrick Hughes, Aliso Viejo, CA (US); James A. Burke, Santa Ana, CA (US)

Correspondence Address: Frank J. Uxa;Stout, Uxa, Buyan & Mullins, LLP, Sutie 300, 4 Venture, Irvine, CA, 92618, US

(73) Assignee: Allergan, Inc., Irvine, CA (US)

(21) Appl. No.: 11/075,200

(22) Filed: Mar. 8, 2005

(51) Int. Cl. A61F 2/00; B29D 11/00

(52) U.S. Cl. 424/427; 264/1.7

(57) ABSTRACT

Drug delivery systems suitable for administration into the interior of an eye of a person or animal are described. The present systems include a drug delivery element that provides directional delivery of one or more drugs to a desired target site in the eye and remain invisible to the individual in which the element is placed. The present drug delivery systems may include an adhesive portion that adheres the drug delivery element to the retina of the eye, and/or a polymeric envelope substantially surrounding the drug delivery element to form an intravitreal implant. The envelope may enhance the stability and handling of the implant when the implant is being placed in the eye. Methods of producing and using the drug delivery systems are also described.

### BACKGROUND

The present invention relates to therapeutic drug delivery systems and methods of manufacturing and for using such systems to treat diseases or disorders of one or more eyes of an individual. More specifically, the present invention relates to intraocular drug delivery systems, structured for placement in the interior of an eye of an individual to treat or reduce one or more symptoms of an ocular condition to improve or maintain vision of a patient without causing substantial toxicity, damage, or injury to intraocular tissues.

The macula is one of the most important parts of the eye. For example, the macula contains the highest density of photoreceptors per unit area in the eye, and the macula provides the visual system with the capability to inspect details and has an important role in stereoscopic (binocular) vision.

Two of the most sight-threatening macular pathologic conditions are sub-foveal choroidal neovascularization (CNV) and macular edema. Usually existing treatment options for both conditions include intraocular delivery of anti-angiogenic agents, such as anti-angiogenic compounds, or anti-inflammatory agents, such as corticosteroids, for prolonged periods of time.

Treatment of macular diseases with traditional pharmacological approaches is difficult. This difficulty can be attributed to one or more of the following: (a) the presence and functioning of the blood-retinal barriers (Cunha-Vaz, J. G., The blood-retinal barriers system. Basic concepts and clinical evaluation. Exp Eye Res, 2004. 78(3): p. 715-21); (b) the relatively large distance form the anterior part of the eye (pars plana), where an insertion and fixation of intraocular drug delivery device (DDD), such as an implant might be uncomplicated and somewhat safe; (c) the relatively small size of the macula (less than 1% of the total retinal surface); and (d) the peculiarities of the structure and biochemical composition of the vitreous body which could affect distribution of certain drugs inside the eye (Sebag, J., Imaging vitreous. Eye, 2002. 16(4): p. 429-39; and Balazs, E. A., Fine structure and function of ocular tissues. The vitreous. Int Ophthalmol Clin, 1973. 13(3): p. 169-87).

Examples of drug delivery devices that attach to the sclera of an eye are disclosed in U.S. Pat. No. 5,466,233 (Weiner) entitled Tack for intraocular drug delivery and method for inserting and removing same; U.S. Pat. No. 5,904,144 (Hamming et al.) entitled Method for treating ophthalmic diseases; U.S. Pat. No. 5,972,369 (Roorda et al.) entitled Diffusional implantable delivery system; U.S. Pat. No. 6,299,895 (Hamming et al.) entitled Device and method for treating ophthalmic diseases; U.S. Pat. No. 6,488,938 (Ogura et al.) entitled Polylactic acid scleral plug; U.S. Patent Publication No. 2002/0026176 (Varner et al.) entitled Devices for intraocular drug delivery; and International Patent Publication No. WO 03/092564 (Allan et al.) entitled Device for delivery of biologically active agents. Existing devices also include those that are transplanted through the pars plana into the anterior vitreous. A device penetrating the retina (retinal tack) is disclosed in U.S. Pat. No. 6,165,192 (Greenberg et al.) entitled Method and apparatus for intraocular retinal tack inserter.

All three approaches, such as the scleral attached devices, anterior vitreous placed devices, and retinal tacks have disadvantages. For example, devices attached to the sclera are positioned away from the macula. More specifically, such devices are anchored to the pars plana. Therefore, it is necessary to provide at least 2-3 times greater vitreal concentration of a therapeutic agent or drug relative to the average therapeutic level of such agents or drugs in order to obtain desired therapeutic levels of the agent or drug in the macula. This requirement is due to the fact that the vitreous is not a well stirred compartment. In the absence of convective forces, considerable concentration gradients can develop within the vitreous. To achieve therapeutic drug levels distal to the device, greater concentrations of therapeutic agents near or proximal to the device are required.

The same issues exist for implants placed into the anterior vitreous. In addition, due to the asymmetric positioning of the device, the adjacent retina receives considerably more amounts of the drug, which increases the probability of different side effects, including toxicity (in some cases, it has been proposed that the retinal receives up to 10 times more of the drug, according to some models (Maurice, D., Review: practical issues in intravitreal drug delivery. J Ocul Pharmacol Ther, 2001. 17(4): p. 393-401)).

Retinal tacks have the disadvantage of penetrating the neural retina (and other tissues like RPE and choroid), which could lead to fibrovascular tissue proliferation arising from the choroid (“foreign body” type reaction) (Daus, W., et al., Histopathology findings following retinal tack implantation. Ophthalmologica, 1989. 199(4): p. 162-4), which could substantially decrease (or eliminate completely) the release of the drug in the long-term.

In addition, implant elements or implants have been described which can be placed in the interior of an eye to release therapeutic agents from the implant and obtain a therapeutic benefit. For example, U.S. Pat. No. 6,713,081 discloses ocular implant devices made from polyvinyl alcohol and used for the delivery of a therapeutic agent to an eye in a controlled and sustained manner. The implants may be placed subconjunctivally or intravitreally in an eye. Biocompatible implants for placement in the eye have also been disclosed in a number of patents, such as U.S. Pat. Nos. 4,521,210; 4,853,224; 4,997,652; 5,164,188; 5,443,505; 5,501,856; 5,766,242; 5,824,072; 5,869,079; 6,074,661; 6,331,313; 6,369,116; and 6,699,493; and U.S. Patent Publication Nos. 2004/0175410 and 2004/0208910.

Thus, there remains a need for new drug delivery systems and methods which may be used to treat ocular conditions by being intraocularly placed in an eye of a patient and which have little or no adverse reactions to the patient receiving the implants. For example, it may be understood that a problem associated with existing intraocular implants is that they may provide undirected drug delivery in the eye and therefore may not provide a therapeutic effect to a desired target region, and that they are visibly noticeable to the individual in which the implant was placed.

### SUMMARY

The present invention addresses this need and provides therapeutic drug delivery systems and methods that provide effective treatment of one or more ocular conditions without causing substantial damage or injury to ocular tissues. The present systems are useful for delivering one or more therapeutic agents or drugs to the interior of an eye of an individual, such as a person or animal. The present systems include a drug delivery element that provides directional delivery of one or more drugs to a desired target site in the eye and remain invisible or clinically unnoticeable to the individual in which the element is placed. For example, the drug delivery element can selectively or directionally deliver or release therapeutically effective amounts of one or more drugs to a region of the retina of an eye, such as the macula of the eye. The drug delivery element remains invisible, or substantially invisible, to the patient receiving the drug delivery element by maintaining the drug delivery element in a fixed, or substantially fixed, position in the eye. In other words, once the drug delivery element is placed at a desired location in the eye, such as in proximity to the macula, the drug delivery element does not significantly move relative to the patient's retina, and therefore, the drug delivery element is unnoticed by the patient, or does not substantially interfere with the patient's vision.

In one broad embodiment, a therapeutic drug delivery system as described herein comprises a polymeric drug delivery element. The drug delivery element comprises a drug-containing reservoir portion and a drug dispensing portion extending from the reservoir portion. One or more dispensing ports are provided on the drug dispensing portion. The dispensing port(s) is effective in providing directional delivery of the drug from the drug delivery element to a desired target location of an eye of an individual. The drug delivery element remains invisible or visually unnoticeable to the individual when the drug delivery element is fixedly positioned in the eye.

The present drug delivery systems may include an adhesive portion that adheres the drug delivery element to the retina of the eye, and/or a polymeric envelope substantially surrounding the drug delivery element to form an intravitreal implant. The envelope may enhance the stability and handling of the implant when the implant is being placed in the eye.

The components of the drug delivery system are preferably formed of biodegradable polymeric materials. Each of the components preferably degrades at different rates so that a desired drug delivery and therapeutic benefit can be obtained.

In one specific embodiment, the drug delivery element comprises a first biodegradable polymeric component, and the drug delivery system further comprises an adhesive portion in contact with the drug delivery element; and an envelope substantially surrounding the drug delivery element to form an intravitreal implant. The envelope comprises a biodegradable polymeric component different than the first biodegradable polymeric component and degrades at a faster rate than the drug delivery element when the implant is placed in the vitreous of the eye. The adhesive portion is effective in affixing the drug delivery element in a substantially fixed position on the retina, such as in proximity to the macula. The drug dispensing port of the drug delivery element may comprise a biodegradable or photosensitive polymeric component effective in controlling release of the drug from the drug delivery element.

In yet another embodiment, a method of treating an ocular condition of an individual person or animal comprises administering the present drug delivery systems to the interior of an eye of the individual, such as the vitreous or posterior segment of the eye.

In a further embodiment, a method of manufacturing a drug delivery system in accordance with the present disclosure comprises forming a polymeric material into a shape of a drug delivery element, as described above, The forming can include one or more steps of extruding the polymeric material, injection molding the polymeric material, and machining the polymeric material.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

Additional aspects and advantages of the present invention are set forth in the following drawings, description and claims.

### BRIEF DESCRIPTION OF THE DRAWINGS

FIG. 1. is a perspective view of a drug delivery element of the present drug delivery systems.

FIG. 2 is a plan view of the dispensing port of the drug delivery element of FIG. 1.

FIG. 3 is a magnified plan view of the dispensing end of the drug dispensing portion of the drug delivery element of FIG. 1.

FIG. 4 is a perspective view of an intravitreal implant including the drug delivery element of FIG. 1

FIG. 5. is a schematic plan view of an ocular fundus diagram showing a potential site of intraocular placement of the present drug delivery system.

FIG. 6 is a sectional view of the diagram of FIG. 5.

### DESCRIPTION

Drug delivery systems and methods have been invented which provide effective treatment of ocular conditions, such as disorders or diseases of the posterior segment of an eye of an individual, such as a human or animal. The present systems and methods provide effective treatment of one or more ocular conditions without causing substantial damage or injury to ocular tissues and without substantially interfering with the individual's vision. The systems comprise materials which are effective in providing extended or sustained release of one or more therapeutic agents or drugs to the interior of an eye of an individual. For example, a single administration of the present systems can deliver or release the drug(s) into the eye for a period of time greater than about one month and can provide effective treatment, such as a reduction or alleviation of one or more symptoms, of ocular conditions for several months or years.

As used herein, a drug delivery system refers to one or more devices, such as a drug delivery device, structured, such as being sized and shaped, for placement in the interior of an eye, such as the posterior segment or vitreous of an eye.

The present systems include a drug delivery element that provides directional delivery of one or more drugs to a desired target site in the eye and remains invisible, or substantially invisible, to the individual in which the element is placed. For example, the drug delivery element can selectively or directionally deliver or release therapeutically effective amounts of one or more drugs to a region of the retina of an eye, such as the macula of the eye. In the context of the present invention, the drug delivery systems can be understood to comprise a therapeutic component that comprises one or more therapeutic agents or drugs. For purposes of convenience, the word “drug” as used herein is intended to refer to a single drug or combinations of drugs, unless specifically stated otherwise.

The drug delivery element of the present systems remains invisible, or substantially invisible, to the patient receiving the drug delivery element by maintaining the drug delivery element in a fixed, or substantially fixed, position in the eye. In other words, once the drug delivery element is placed at a desired location in the eye, such as in proximity to the macula, the drug delivery element does not significantly move relative to the patient's retina, and therefore, the drug delivery element is unnoticed by the patient, or does not substantially interfere with the patient's vision. Since there is little or no movement of the element when placed in the eye, there is no visual perception that the element is actually in the eye.

The present systems comprise solid, semi-solid, or substantially solid polymeric elements. The present drug delivery systems can be provided in a liquid composition if desired, and thus, the present invention encompasses compositions which may comprise the drug delivery systems disclosed herein and a liquid or aqueous carrier component.

Examples of the present drug delivery systems are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, front, backward, forward, anterior, posterior, proximal, and distal are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims.

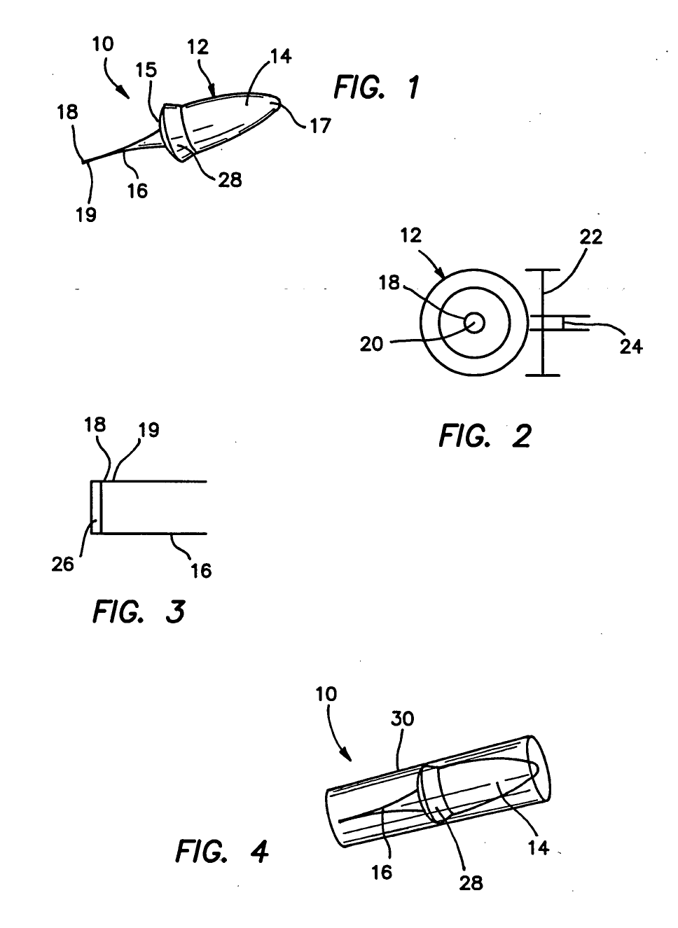

FIG. 1 is an illustration of a therapeutic drug delivery system 10 that is useful for placement into the posterior segment of an eye of an individual. In other words, the drug delivery system 10 is structured, such as sized and/or shaped, to be placed into the posterior segment of the eye to provide a desired therapeutic effect. The drug delivery system 10 comprises a polymeric drug delivery element 12. The drug delivery element 12 comprises a therapeutic component, such as one or more drugs, as discussed herein. In reference to the drawings, the drug delivery element 12 comprises a drug-containing reservoir portion 14 and a drug dispensing portion 16.

The drug dispensing portion 16 extends from the reservoir portion 14. Thus, as illustrated, the drug dispensing portion 16 extends from the first reservoir end 15. The drug dispensing portion 16 provides a drug delivery path from the reservoir portion 14 to a drug dispensing port 18. The drug dispensing portion 16 is preferably permanently attached to the reservoir portion 14, and more preferably is integrally formed with the reservoir portion 14. However, in certain embodiments, the drug dispensing portion 16 is non-permanently attached to the reservoir portion 14.

The drug dispensing portion 16 has a dispensing port 18 located on the drug dispensing portion 16 to provide directional delivery of the drug from the drug delivery element 12 to a desired target location of an eye of an individual, such as the retina, and more specifically the macula of the eye. As shown in the drawings, the dispensing port 18 is located at the end or terminal region 19 of the dispensing portion 16. In other embodiments, the dispensing port 18 may be provided at a location between the end 19 of the dispensing portion 16 and the first end 15 of the reservoir portion 14. In still further embodiments, the dispensing portion 16 may have two or more dispensing ports 18 located along the length of the dispensing portion 16. Such configurations can be selected based on the ocular condition(s) desired to be treated.

The dispensing port 18 provides directional delivery of the drug present in the drug delivery element 12. The dispensing port 18 may be understood to be an opening or aperture in the dispensing portion 16, or the dispensing port 18 may comprise a drug releasing surface 20, as shown in FIG. 2 that releases drug preferentially from the dispensing port 18 relative to other regions of the drug delivery element 12. For example, in embodiments of the present systems which include a chamber or cavity in the reservoir portion 14, the dispensing portion 16 may include a conduit extending along the length of the dispensing portion 16. Drug that is present in the chamber or cavity may pass through the conduit and out of the dispensing port 18 into the eye. In embodiments of the present systems in which the drug delivery element 12 primarily comprises a matrix of a polymeric component and drug, the material of the dispensing port 18 may degrade or release the drug more quickly than the drug is released from the rest of the drug delivery element 12.

As stated above, the reservoir portion 14 is illustrated as having a first reservoir end 15 and a second reservoir end 17. In the illustrated embodiment, the second reservoir end 17 has a maximum cross-sectional distance (e.g., diameter) less than the maximum cross-sectional distance (e.g., diameter) of the first reservoir end 15. In certain embodiments, the reservoir portion 14 has a maximum cross-sectional distance (e.g., a diameter) of about 400 micrometers or less. For example, some preferred embodiments may have a maximum cross-sectional diameter of about 300 micrometers.

The dispensing port 18 is provided at a location of the drug dispensing portion 16 that has a small maximum cross-sectional distance 24, as shown in FIG. 2. Advantageously, the maximum cross-sectional distance 24 is effective in obstructing few photoreceptor cells of the individual's retina. For example, the maximum cross-sectional distance, such as the diameter, of the drug dispensing portion 16 at a region or location where the dispensing port 18 is located, such as at the end 19, shown in FIG. 1 and FIG. 3, obstructs fewer than about 5 cones of the individual's retina. For example, the dispensing portion 16 at the dispensing port region obstructs only between about 2 and about 4 cones of the retina. As a further example, the dispensing port region may have a maximum cross-sectional distance, such as a diameter, from about 20 micrometers to about 50 micrometers. A relationship between the maximum diameter 24 of the dispensing port region of the dispensing portion 16 and the maximum diameter 22 of drug delivery element 12 is illustrated in FIG. 2.

As shown in FIG. 3, and as discussed herein, the drug dispensing portion 16 may comprise a cover or membrane 26 located around the dispending port 18. The cover or membrane 26 is formed of a material, such as a polymeric material, that can be removed relatively quickly when the drug delivery element 12 is placed in an eye. For example, the cover 26 can be removed before the reservoir portion 14 degrades, and thus, a directional drug delivery path can be established from the drug delivery element 12 into the eye. In that regard, the material from which the cover 26 is formed may be understood to have a higher diffusive resistance (permeability) compared to the material of the drug delivery element 12, or more specifically, the reservoir portion 14. Preferably, the cover material is biodegradable or bioerodible. Thus, it may be understood that cover 26 comprises a biodegradable polymeric component with different degradation properties, such as degradation rate, than the material of the drug delivery element 12. It may also be understood that the dispensing port 18 comprises a biodegradable or removable polymeric component. In certain embodiments, the cover 26 comprises a photosensitive material, such as a photosensitive polymeric material. Before placement in the eye, the cover 26 effectively prevents drug from being released from the drug delivery element 12. However when placed in the eye, the cover 26 is relatively quickly removed, such as before the drug delivery element 12 is degraded, to provide a directional drug delivery path for the drug provided in the drug delivery element 12. When a photosensitive polymeric material is used, the cover 26 can be removed by light passing through the individual's eye and reacting with the cover 26.

The present drug delivery systems 10, including the drug delivery element 12, are desirably substantially or completely invisible to the individual in which the system or element is placed. For example, the element 12 may be retained in a fixed position in the eye so that the element 12 does not visibly move relative to the retina. By fixedly positioning the element 12 in the eye, the element 12 cannot be seen or is undetectable by the individual.

To fixedly position the drug delivery element 12 in the eye, the drug delivery system 10 may further comprise an adhesive portion or adhesive component 28, as shown in FIG. 1. The adhesive portion 28 is provided on the drug delivery element 12 and is effective in adhering the drug delivery element to an interior region of the eye, such as the retina. Importantly, the drug delivery element 12 can be adhered to the retina without injuring the retinal tissue or retinal cells. For example, the adhesive portion enables the drug delivery element 12 to be affixed or attached to the retina, or other intraocular tissue, without being inserted in a slit or wall of the retinal or tissue. Thus, the adhesive portion 28 is effective in fixing or stabilizing the drug delivery element 12 on the surface of the retina and is effective in reducing a movement sensation associated with potential movement of the drug delivery element 12 relative to the retina. In embodiments in which the dispensing port 18 is to be placed above the macula, the adhesive portion 28 is effective in maintaining the dispensing port 18 in that position as the drug is released from the dispensing portion 16.

The adhesive portion 28 is illustrated as a continuous band circumscribing a portion of the drug delivery element 12. More specifically, the adhesive portion 28 is illustrated as circumscribing a fraction of the reservoir portion 14 of the drug delivery element 12. Although the illustrated embodiment shows the adhesive portion as a continuous band, other embodiments may comprise an adhesive portion that is segmented. For example, the adhesive portion 28 may be provided in two or more segments located around the drug delivery element 12. Any suitable material may be used to adhere the drug delivery element to an intraocular region of the eye. In the illustrated embodiment, the adhesive portion 28 comprises a hydrogel material, such as a polyethylene glycol (PEG) material. One specific example is SS-PEG available from Nektar Therapeutics (San Carlos, Calif.) (Margalit, E., et al., Bioadhesives for intraocular use. Retina, 2000. 20(5): p. 469-77).

The drug delivery element 12 can comprise a variety of materials that are ophthalmically acceptable and useful for delivering drugs for extended periods of time. In one embodiment, such as the illustrated embodiment, the drug delivery element 12 comprises a biodegradable polymeric component. The biodegradable polymeric component degrades or erodes at an intermediate rate relative to other components of the drug delivery elements disclosed herein. In certain embodiments, the biodegradable polymeric component substantially completely degrades after about 3 months from when the drug delivery element 12 is placed in the eye. In such embodiments, the drug delivery element 12 comprises, consists essentially of, or consists entirely of, a matrix of one or more biodegradable polymers and one or more drugs. In other words, the drug(s) are substantially distributed throughout the matrix and accordingly, the drug delivery element 12.

The present drug delivery systems 10 may also comprise a biodegradable envelope 30, as shown in FIG. 1, that substantially surrounds the drug delivery element 12. In the context of the present description, the combination of the drug delivery element 12 and the envelope 30 may be understood to be an intravitreal implant. For example, an intravitreal implant that comprises the drug delivery element 12 and the envelope 30 can be placed into the interior of an eye of an individual. In the illustrated embodiment, the envelope 30 is effective in enhancing the stability of the intravitreal implant when the implant is placed in the interior of the eye, such as in the vitreous of the eye.

In the illustrated embodiment, the envelope 30 comprises a rapidly degrading biodegradable polymer. For example, the envelope 30 is formed of a material that degrades quickly after placement in the eye. In preferred embodiments, the envelope 30 degrades before both the cover 26 of the dispensing port 18 and before the drug delivery element 12. The envelope 30 of the illustrated embodiment has a maximum expected life in the vitreous less than about 24 hours. Any suitable polymer may be used in producing the envelope 30. One example includes Gantrez type polymers available from International Specialty Products (Wayne, N.J.). The envelope 30 has a maximum cross-sectional distance, such as a diameter, that is substantially equal to the maximum cross-sectional distance of the drug delivery element 12. Thus, with respect to certain embodiments described herein, the envelope may have a maximum cross-sectional diameter of about 400 micrometers. In other preferred embodiments, the envelope has a maximum cross-sectional diameter of about 300 micrometers or less.

The intravitreal implant may be provided in any appropriate geometric form. For example, the intravitreal implant may be provided in a shape selected from the group consisting of cylinders, triangles, rectangles, and other shapes with multiple walls. As shown in FIG. 4, the intravitreal implant has a cylindrical shape.

In context of the present description, one preferred embodiment of the present drug delivery systems comprises a biodegradable drug delivery element which comprises a drug-containing reservoir portion and a drug dispensing portion having a drug dispensing port covered by a biodegradable polymeric cover; an adhesive portion in contact with the drug delivery element and effective in adhering the drug delivery element to a retinal surface; and a biodegradable envelope substantially surrounding the drug delivery element. Thus, it may be understood that the drug delivery element comprises a first biodegradable polymeric component; the dispensing port comprises a different second biodegradable polymeric component; and the envelope comprises a different third biodegradable polymeric component. In this embodiment, the envelope substantially completely degrades before the dispensing port cover and the drug delivery element, and the dispensing port cover substantially completely degrades before the drug delivery element. It will be understood that there may be some overlap as to when particular components of the drug delivery systems begin and finish degrading. However, as a general rule, the envelope comprises a material that degrades more quickly than the cover and drug delivery element, and the cover comprises a material that degrades more quickly than the drug delivery element.

The drug reservoir portion 14 of the foregoing embodiment may comprise a polymeric component effective in delivering or releasing the drug to the macula or posterior of the eye for a period of time from about 3 months to about 2 years after the drug delivery system 10 is placed in the eye of the individual. The envelope 30 of the foregoing embodiment may comprise a polymeric component effective in forming an intravitreal implant having physical properties to facilitate injection of the implant into the vitreous. For example, the implant may have improved rigidity, flexibility, or reduced friction relative to the drug delivery element. The envelope may degrade within about 24 hours after placement in the vitreous of the eye. The dispensing port cover 26 may substantially completely degrade between about 24 hours and 3 months after placement in the eye.

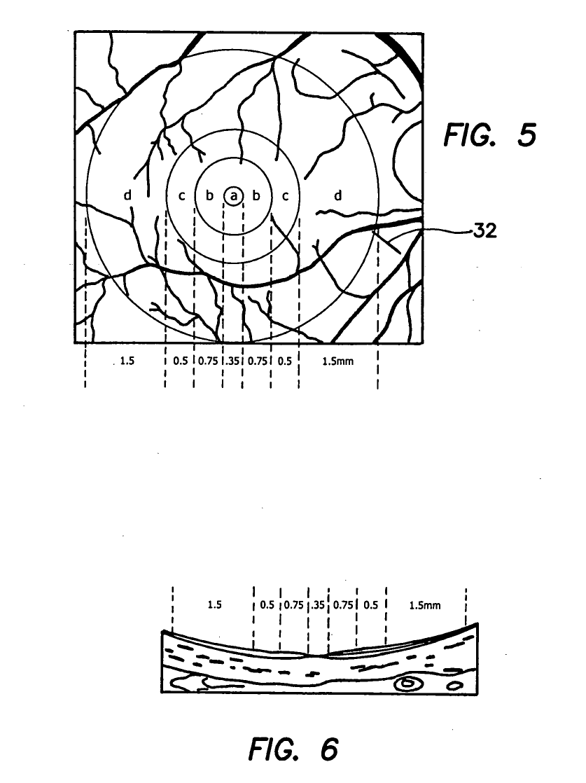

As discussed herein, the dispensing portion 16 of the foregoing embodiment has a small maximum cross-sectional distance. Preferably, the dispensing portion 16, as well as the entire drug delivery element 12, is clinically unnoticeable by the individual in which the implant is placed. In other words, the drug delivery element, including the drug dispensing portion 16, is invisible to the patient. To reduce the visibility of the present drug delivery systems, the dispensing portion of the drug delivery element can be oriented with respect to the reservoir portion such that the reservoir portion would be no closer than about 15 degrees (e.g., about 4.5 mm) from the fovea when the dispensing port is placed in proximity to the macula. At that distance, the visual acuity drops to ˜10 to 15% of the best corrected central visual acuity (Weymouth, F. W., Visual acuity within the area centralis and its relation to eye movements and fixation. Am J Ophthalmol, 1928.11: p. 947-950). In addition, as discussed herein, the presence of a specialized vitreous membrane located in front of the macula, (bursa premacularis (Jongebloed, W. L. and J. F. Worst, The cisternal anatomy of the vitreous body. Doc Ophthalmol, 1987. 67(1-2): p. 183-96)) may be taken into consideration. As discussed herein, in certain embodiments, the dispensing port is inserted through this membrane. Therefore, one suitable placement site 32 of the drug delivery system can be at the nasal-inferior side of the macula close to the optic disc, as shown in FIG. 5.

Movement of any object larger than a few microns in front of the retina would create a perception for the event and, therefore would be undesirable. Thus, in one embodiment the implant has an adhesive portion that would ensure the fixation of the object on the surface of the retina. A fixed object positioned towards the retina would not create a movement sensation and would perceptually disappear (Heckenmueller, E. G., Stabilization of the Retinal Image: A Review of Method, Effects, and Theory. Psychol Bull, 1965. 63: p. 157-69). In addition, the small size of the drug dispensing portion would obstruct visually only few (2-4) cones, and, therefore, would create only a very small defect in the existing visual field.

As discussed herein, the present drug delivery systems comprise one or more drugs. The drug is provided in an amount effective in providing a desired therapeutic effect to an individual, such as a human or animal patient, when the system is administered to the interior of an eye of the individual and the drug is released therefrom. It may be understood that the present systems are useful for injection or implantation into the interior of an eye of the individual. More specifically, the present systems are useful for injection or implantation or other administration technique into the posterior segment of the eye, such as into the vitreous of an eye.

Examples of drugs useful in the present drug delivery systems include chemical compounds, macromolecules, proteins, and the like, which are effective in treating an ocular condition, such as an ocular condition of the posterior segment of an eye.

Drugs which may be provided in the present drug delivery systems may be obtained from public sources or may be synthesized using routine chemical procedures known to persons of ordinary skill in the art. Drugs are screened for therapeutic efficacy using conventional assays known to persons of ordinary skill in the art. For example, drugs can be monitored for their effects on reducing intraocular pressure, reducing or preventing neovascularization in the eye, reducing inflammation in the eye, and the like using such conventional assays. Thus, the present systems can comprise a variety of drugs.

Specific examples of drugs useful in the present systems include one or more of the following: anti-excitotoxic agents, anti-histamine agents, antibiotic agents, beta blocker agents, one or more steroid agents, anti-neoplastic agents, ocular hemorrhage treatment agents, immunosuppressive agents, anti-viral agents, anti-oxidant agents, anti-inflammatory agents, including non-steroidal antiinflammatory agents, adrenergic receptor agonists and antagonists, VEGF inhibitor agents, neuroprotective agents, and any ophthalmically therapeutic macromolecule that can be identified and/or obtained using routine chemical screening and synthesis techniques.

The drug delivery system may also include salts of the drugs. Pharmaceutically acceptable acid addition salts of therapeutic compounds of the present systems are those formed from acids which form non-toxic addition salts containing pharmaceutically acceptable anions, such as the hydrochloride, hydrobromide, hydroiodide, sulfate, or bisulfate, phosphate or acid phosphate, acetate, maleate, fumarate, oxalate, lactate, tartrate, citrate, gluconate, saccharate and p-toluene sulphonate salts. Based on the disclosure herein, it may be understood that the drugs are ophthalmically acceptable.

Examples of antihistamines include, and are not limited to, loradatine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, chlorcyclizine, thonzylamine, and derivatives thereof.

As used herein, the term “derivative” refers to any substance which is sufficiently structurally similar to the material of which it is identified as a derivative so as to have substantially similar functionality or activity, for example, therapeutic effectiveness, as the material when the substance is used in place of the material.

Examples of antibiotics include without limitation, cefazolin, cephradine, cefaclor, cephapirin, ceftizoxime, cefoperazone, cefotetan, cefutoxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof.

Examples of beta blockers include acebutolol, atenolol, labetalol, metoprolol, propranolol, timolol, and derivatives thereof.

Examples of steroids include corticosteroids, such as cortisone, prednisolone, flurometholone, dexamethasone, medrysone, loteprednol, fluazacort, hydrocortisone, prednisone, betamethasone, prednisone, methylprednisolone, triamcinolone hexacatonide, paramethasone acetate, diflorasone, fluocinonide, fluocinolone, triamcinolone, triamcinolone acetonide, derivatives thereof, and mixtures thereof.

Examples of antineoplastic agents include adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof.

Examples of immunosuppresive agents include cyclosporine, azathioprine, tacrolimus, and derivatives thereof.

Examples of antiviral agents include interferon gamma, zidovudine, amantadine hydrochloride, ribavirin, acyclovir, valciclovir, dideoxycytidine, phosphonoformic acid, ganciclovir and derivatives thereof.

Examples of antioxidant agents include ascorbate, alpha-tocopherol, mannitol, reduced glutathione, various carotenoids, cysteine, uric acid, taurine, tyrosine, superoxide dismutase, lutein, zeaxanthin, cryotpxanthin, astazanthin, lycopene, N-acetyl-cysteine, carnosine, gamma-glutamylcysteine, quercitin, lactoferrin, dihydrolipoic acid, citrate, Ginkgo Biloba extract, tea catechins, bilberry extract, vitamins E or esters of vitamin E, retinyl palmitate, and derivatives thereof.

Some additional examples of drugs include anacortave (anti-angiogenesis compound), hyaluronic acid (ocular hemorrhage treatment compound), ketorlac tromethamine (Acular) (non-steroidal anti-inflammatory agent), ranibizumab, pegaptanib (Macugen) (VEGF inhibitors), cyclosporine, gatifloxacin, ofloxacin, epinastine (antibiotics). Macromolecules useful in the present implants may have a molecular weight greater than about 1000 Daltons, for example between about 10,000 and about 1 million Daltons. Examples of suitable macromolecules include large proteins.

Other drugs include squalamine, carbonic anhydrase inhibitors, brimonidine, prostamides, prostaglandins, antiparasitics, antifungals, tyrosine kinase inhibitors, glutamate receptor antagonists, including NMDA receptor antagonists, and derivatives thereof.

In view of the foregoing, it can be appreciated that the present drug delivery systems can comprise many different types of drugs, and that such agents are routinely known to or obtained by persons of ordinary skill in the art.

The drug may be in a particulate or powder form and may be associated with the polymeric component of the drug delivery element in a number of different configurations. For example, drug particles may be entrapped by a polymer matrix, such as a biodegradable polymer matrix. Or, drug particles may be encompassed by the polymeric component, such as in the form of a diffusion controlled implant.

The drug of the present drug delivery systems is preferably present in an amount from about 10% to 90% by weight of the drug delivery system or drug delivery element. More preferably, the drug is present in an amount from about 20% to about 80% by weight of the system or element. In a preferred embodiment, the drug comprises about 40% by weight of the system or element (e.g., 30%-50%). In another embodiment, the drug comprises about 60% by weight of the system or element.

Suitable polymeric materials or compositions for use in the drug delivery systems include those materials which are compatible, that is biocompatible, with the eye so as to cause no substantial interference with the functioning or physiology of the eye. In certain embodiments, the materials preferably are at least partially and more preferably substantially completely biodegradable or bioerodible. In other embodiments, non-biodegradable polymers are used. Non-biodegradable polymers may be particularly useful in diffusion-based drug delivery systems, such as systems which include a drug-containing core and have a coating with a dispensing port to permit the drug to diffuse therefrom.

Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present implants.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate.

Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyesters, polyethers and combinations thereof which are biocompatible and may be biodegradable and/or bioerodible.

Some preferred characteristics of the polymers or polymeric materials for use in the present invention may include biocompatibility, compatibility with the therapeutic component, ease of use of the polymer in making the drug delivery systems of the present invention, a half-life in the physiological environment of at least about 6 hours, preferably greater than about one day, not significantly increasing the viscosity of the vitreous, and water insolubility.

The biodegradable polymeric materials which are included to form the present elements are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended release profile of the implant is the relative average molecular weight of the polymeric composition employed in the implant. Different molecular weights of the same or different polymeric compositions may be included in the implant to modulate the release profile. In certain drug delivery systems, the relative average molecular weight of the polymer will range from about 9 to about 64 kD, usually from about 10 to about 54 kD, and more usually from about 12 to about 45 kD.

In some systems, copolymers of glycolic acid and lactic acid are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the drug delivery element, where a more flexible element is desirable for larger geometries. The % of polylactic acid in the polylactic acid polyglycolic acid (PLGA) copolymer can be 0-100%, preferably about 15-85%, more preferably about 35-65%. In some elements, a 50/50 PLGA copolymer is used.

The biodegradable polymer matrix of some drug delivery systems may comprise a mixture of two or more biodegradable polymers. For example, the elements of the system may comprise a mixture of a first biodegradable polymer and a different second biodegradable polymer. One or more of the biodegradable polymers may have terminal acid groups.

Release of a drug from an erodible polymer is the consequence of several mechanisms or combinations of mechanisms. Some of these mechanisms include desorption from the implants surface, dissolution, diffusion through porous channels of the hydrated polymer and erosion. Erosion can be bulk or surface or a combination of both. As discussed herein, a matrix of the drug delivery system may release drug at a rate effective to sustain release of an amount of the therapeutic agent for more than one week after implantation into an eye. In certain systems, therapeutic amounts of the therapeutic agent are released for more than about one month, and even for about six months or more.

The release of the drug from a drug delivery system comprising a biodegradable polymer matrix may include an initial burst of release followed by a gradual increase in the amount of the drug released, or the release may include an initial delay in release of the therapeutic agent followed by an increase in release. When the biodegradable system is substantially completely degraded, the percent of the therapeutic agent that has been released is about one hundred. Compared to existing implants, the systems disclosed herein do not completely release, or release about 100% of the therapeutic agent, until after about one week of being placed in an eye.

It may be desirable to provide a relatively constant rate of release of the therapeutic agent from the system over the life of the system. For example, it may be desirable for the therapeutic agent to be released in amounts from about 0.01 μg to about 2 μg per day for the life of the system. However, the release rate may change to either increase or decrease depending on the formulation of the biodegradable polymer matrix. In addition, the release profile of the therapeutic agent may include one or more linear portions and/or one or more non-linear portions. Preferably, the release rate is greater than zero once the implant has begun to degrade or erode.

The present drug delivery elements may be monolithic, i.e. having the active agent or agents homogenously distributed through a polymeric matrix, or encapsulated, where a reservoir of active agent is encapsulated by a polymeric matrix, as discussed herein. For example, the reservoir portion of the drug delivery element may comprise a matrix of the drug and the polymeric component, or it may comprise a chamber containing the drug in powder or liquid form surrounded by a polymeric material. Due to ease of manufacture, monolithic elements are usually preferred over encapsulated forms. However, the greater control afforded by the encapsulated, reservoir-type elements may be of benefit in some circumstances, where the therapeutic level of the drug falls within a narrow window. In addition, the therapeutic component, including the therapeutic agent(s) described herein, may be distributed in a non-homogenous pattern in a polymeric matrix. For example, the drug delivery element may include a portion that has a greater concentration of the therapeutic agent relative to a second portion of the element.

Thus, it may be understood that bioerodible polymers can be used to form monolithic homogeneous or heterogeneous implants and microparticulates, membrane controlled implants or microparticulates, multistage delivery systems, or any combination thereof. The polymers comprising the carrier delivery system can be natural or synthetic polymers. In certain embodiments, examples of polymers include polyesters, poly(ortho esters) or polyanhydrides, as discussed above. Some specific polymers include poly-lactic acid (PLA), poly(lactide-co-glycolide) (PLGA), poly-l-lactic acid (PLLA), polycaprolactone, poly(ortho acetate), and combinations thereof.

The proportions of therapeutic agent, polymer, and any other modifiers may be empirically determined by formulating several drug delivery elements with varying proportions. A USP approved method for dissolution or release test can be used to measure the rate of release (USP 23; NF 18 (1995) pp. 1790-1798). For example, using the infinite sink method, a weighed sample of the element is added to a measured volume of a solution containing 0.9% NaCl in water, where the solution volume will be such that the drug concentration after release is less than 5% of saturation. The mixture is maintained at 37° C. and stirred slowly to maintain the elements in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

In addition to the drug, the drug delivery systems disclosed herein may include effective amounts of solubility enhancing agents, such as cyclodextrins and cyclodextrin derivatives, buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such the buffering agent may be as much as about 5% by weight of the total implant. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, phenylethanol and the like and mixtures thereof. These agents may be present in amounts of from 0.001 to about 5% by weight and preferably 0.01 to about 2% by weight.

The release profile and other characteristics of the present systems can be measured in environments which mimic the vitreous of an eye using conventional methods which are routine to persons of ordinary skill in the art. For example, implants can be immersed in a 3 mL volume of liquid, and the release rate of the therapeutic component can be monitored, as discussed herein.

Various techniques may be employed to produce the drug delivery systems described herein. Useful techniques include, but are not necessarily limited to, solvent evaporation methods, phase separation methods, interfacial methods, molding methods, injection molding methods, extrusion methods, co-extrusion methods, carver press method, die cutting methods, heat compression, combinations thereof and the like.

Specific methods are discussed in U.S. Pat. No. 4,997,652. Extrusion methods may be used to avoid the need for solvents in manufacturing. When using extrusion methods, the polymer and drug are chosen so as to be stable at the temperatures required for manufacturing, usually at least about 85 degrees Celsius. Extrusion methods use temperatures of about 25 degrees C. to about 150 degrees C., more preferably about 65 degrees C. to about 130 degrees C. A drug delivery system may be produced by bringing the temperature to about 60 degrees C. to about 150 degrees C. for drug/polymer mixing, such as about 130 degrees C., for a time period of about 0 to 1 hour, 0 to 30 minutes, or 5-15 minutes. For example, a time period may be about 10 minutes, preferably about 0 to 5 min. The systems are then extruded at a temperature of about 60 degrees C. to about 130 degrees C., such as about 75 degrees C.

In addition, the system may be produced by coextruding so that a coating is formed over a core region during the manufacture of the system.

Compression methods may be used to make the system, and typically yield drug delivery elements with faster release rates than extrusion methods. Compression methods may use pressures of about 50-150 psi, more preferably about 70-80 psi, even more preferably about 76 psi, and use temperatures of about 0 degrees C. to about 115 degrees C., more preferably about 25 degrees C.

In accordance with the disclosure herein, a method of manufacturing the present therapeutic drug delivery system comprises forming a polymeric material into a drug delivery element comprising a drug-containing reservoir portion and a drug dispensing portion extending from the reservoir portion, the drug dispensing portion having a dispensing port effective in providing directional delivery of the drug from the drug delivery element to a desired target location of an eye of an individual when the drug delivery element is fixedly positioned in the eye and is invisible to the individual. The polymeric material may comprise one or more drugs, and the forming step is effective in producing a matrix of the polymeric material and the drug, wherein the drug is substantially distributed throughout the matrix. The forming step of the foregoing method may comprises one or more steps of extruding the material, injection molding the material, compression molding the material, forming tablets (tableting) of the material, and machining the material into a drug delivery element. It may be understood that machining steps may be performed after the polymeric material has been formed into a desired shape that provides directed drug delivery.

The method may also comprise one or more steps of forming an adhesive portion to contact the drug delivery element, and/or forming a polymeric envelop to substantially surround the drug delivery element. In addition, the method may comprise providing a second polymeric material on the dispensing port, wherein the second polymeric material is removable from the dispensing port when the drug delivery element is located in the eye.

The present invention also provides methods of treating one or more ocular conditions of the posterior segment of an eye of an individual. Such methods comprise a step of placing the present drug delivery systems into the posterior segment of the eye of the individual. The drug delivery systems can be placed in the eye using any conventional technique or method known by persons of ordinary skill in the art. In certain embodiments, the drug delivery systems are injected through a needle or similar cannulated device into the vitreous of the eye. For example, the present drug delivery systems can be inserted into the eye by passing the implant through a cannula or needle having a maximum size of 23 gauge. In other words, the drug delivery system or drug delivery element can be inserted into the eye using a 23 gauge or smaller needle or cannula.

As discussed herein, when the target site is the macula of the eye, the drug delivery system is placed near the macula so that the dispensing port selectively delivers the drug to the macula. One potential location for placement of the present drug delivery system is illustrated by line 32 in FIG. 5. For example, the method may comprise a step of inserting the dispensing port of the dispensing portion through the bursa premacularis of the eye (i.e., the vitreous membrane covering the macula). In certain embodiments, the intravitreal implant is placed at nasal-inferior side of the macula close to the optic disc. The dispensing portion of the present drug delivery element can be oriented relative to the drug-containing reservoir so that when the dispensing port is located over the macula, or in proximity to the macula, the reservoir portion is peripherally located relative to the macula. The methods of treating an ocular condition may also comprise a step of affixing, such as adhering, the drug delivery element to a retinal surface.

The present systems are placeable into the interior of an eye of an individual without causing significant adverse effects related to the presence of the systems. For example, the present systems preferably do not cause substantial changes in intraocular pressure of the eye resulting from the placement of the system into the eye. In addition, the present systems preferably do not interfere with the vision of the individual receiving the systems, as discussed herein. For example, the present systems may be optically clear, or may be sized or shaped to be placed in the eye without interfering with the field of vision of the individual. As discussed herein, the present systems are invisible to the patient since the systems are retained in a fixed position relative to the retina of the eye in which the systems are placed.

The drug delivery systems disclosed herein may be placed in the interior of any eye using any suitable device, such as a trocar and the like, or the systems may be administered into the eye in an injectable composition. Therefore, it may be understood that the present invention also encompasses compositions which may contain the present drug delivery systems. The drug delivery systems and/or compositions containing such systems are preferably sterile prior to administration to a patient.

The drug delivery elements of the present systems may be inserted into the eye, for example the vitreous chamber of the eye, by a variety of methods, including placement by forceps or by trocar following making preferably a 0.5 mm (self-sealing) incision in the sclera, or a bigger (up to 3 mm) incision with the need of surgical closure of the wound after the procedure. One example of a device that may be used to insert the elements into an eye is disclosed in U.S. Patent Publication No. 20050033272 The location of the element may influence the concentration gradients of therapeutic component or drug surrounding the element, and thus influence the release rates to the treated area (e.g., an element placed in the upper part of the macula could deliver more drug compared to a device placed in the lower part of the macula).

The present elements are configured to release an amount of the therapeutic agent effective to treat or reduce a symptom of an ocular condition, such as an ocular condition such as glaucoma. More specifically, the elements and the systems comprising such elements, may be used in a method to treat or reduce one or more symptoms of glaucoma or proliferative vitreoretinopathy.

The elements and systems disclosed herein may also be configured to release additional therapeutic agents, as described above, which are effective in treating one or more symptoms of an ocular condition or are effective in preventing diseases or conditions of the eye, such as the following:

MACULOPATHIES/RETINAL DEGENERATION: Non-Exudative Age Related Macular Degeneration (ARMD), Exudative Age Related Macular Degeneration (ARMD), Choroidal Neovascularization, Diabetic Retinopathy, Acute Macular Neuroretinopathy, Central Serous Chorioretinopathy, Cystoid Macular Edema, Diabetic Macular Edema.

UVEITIS/RETINITIS/CHOROIDITIS: Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis and Uveitis Syndrome, Vogt-Koyanagi-Harada Syndrome.

VASCULAR DISEASES/EXUDATIVE DISEASES: Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angitis, Sickle Cell Retinopathy and other Hemoglobinopathies, Angioid Streaks, Familial Exudative Vitreoretinopathy, Eales Disease.

TRAUMATIC/SURGICAL: Sympathetic Ophthalmia, Uveitic Retinal Disease, Retinal Detachment, Trauma, Laser, PDT, Photocoagulation, Hypoperfusion During Surgery, Radiation Retinopathy, Bone Marrow Transplant Retinopathy.

PROLIFERATIVE DISORDERS: Proliferative Vitreal Retinopathy and Epiretinal Membranes, Proliferative Diabetic Retinopathy.

INFECTIOUS DISORDERS: Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis.

GENETIC DISORDERS: Retinitis Pigmentosa, Systemic Disorders with Accosiated Retinal Dystrophies, Congenital Stationary Night Blindness, Cone Dystrophies, Stargardt's Disease and Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum.

RETINAL TEARS/HOLES: Retinal Detachment, Macular Hole, Giant Retinal Tear.

TUMORS: Retinal Disease Associated with Tumors, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors.

MISCELLANEOUS: Punctate Inner Choroidopathy, Acute Posterior Multifocal Placoid Pigment Epitheliopathy, Myopic Retinal Degeneration, Acute Retinal Pigement Epithelitis and the like.

Thus, the present drug delivery systems can be administered to an individual, such as a person or animal, to treat one or more ocular conditions. Thus, the present invention relates to methods of treating a posterior segment ocular condition or conditions.

Although the present invention has been described in detail with regard to certain preferred systems and methods, other embodiments, versions, and modifications within the scope of the present invention are possible. For example, combination therapies are also provided with the present systems. As one example, the present systems may comprise a combination of an anti-inflammatory agent, such as a steroid, and an intraocular pressure reducing agent, such as an alpha-2-adrenergic agonist, to reduce inflammation and intraocular pressure substantially at the same time. Another example, includes a system comprising an anti-excitotoxic agent which may be used as a neuroprotectant, and an anti-inflammatory agents. Combination therapies may use any and all possible combinations of therapeutic agents disclosed herein so long as such combinations are not mutually exclusive.

Other examples of the present implants may include non-biodegradable polymeric components. For example, a non-biodegradable polymeric coating may be provided around a portion or all of the drug containing reservoir. Although biodegradable drug delivery elements may be preferred, elements which include non-biodegradable portions can be effectively used since the drug delivery element can remain attached to the retina.

In addition, the drug delivery element may include a drug delivery rate modifier. Such a modifier can be effective in enhancing the release or decreasing the rate of release of the drug from the drug delivery element. In certain embodiments, the modifier may be photosensitive and controlled by different effects of light interacting with the modifier and/or element.

The present invention also includes within its scope the use of a therapeutic component, such as one or more therapeutic agents or drugs, and one or more biodegradable polymers in the preparation of drug delivery systems for the treatment of an ocular condition, such as a disease or disorder of the posterior segment of an eye, by administration of the system to the interior of an eye, which provide directional delivery of the therapeutic component to a target site in the eye, and remain invisible to the individual in which the system(s) is placed.

All references, articles, patents, applications and publications set forth above are incorporated herein by reference in their entireties.

While this invention has been described with respect to various specific examples and embodiments, it is to be understood that the invention is not limited thereto and that it can be variously practiced within the scope of the following claims.

What is claimed is:

1. A therapeutic drug delivery system useful for placement into a posterior segment of an eye of an individual, comprising:
a polymeric drug delivery element comprising a drug-containing reservoir portion and a drug dispensing portion extending from the reservoir portion, the drug dispensing portion having a dispensing port effective in providing directional delivery of the drug from the drug delivery element to a desired target location of an eye of an individual when the drug delivery element is fixedly positioned in the eye and is invisible to the individual.

2. The system of claim 1, wherein the drug delivery element comprises a biodegradable polymeric component that substantially completely degrades at a time greater than about 3 months after the element is placed in the eye.

3. The system of claim 1, wherein reservoir portion has a maximum cross-sectional distance less than about 400 micrometers.

4. The system of claim 1, wherein the dispensing port is provided at a location of drug dispensing portion having a maximum cross-sectional distance that obstructs less than about 5 cones of the individual's retina.

5. The system of claim 1, wherein the drug delivery element comprises a first biodegradable polymeric component, and the dispensing port comprises a different second biodegradable polymeric component comprising a material that degrades more quickly relative to the first biodegradable polymeric component.

6. The system of claim 1, wherein the drug delivery element is structured to be placed in the eye in proximity to the macula of the eye so that the drug can be directionally delivered to the macula.

7. The system of claim 1, wherein the dispensing portion is oriented with respect to the reservoir portion such that the reservoir portion would be no closer than about 15 degrees from the fovea when the dispensing port is placed in proximity to the macula.

8. The system of claim 1, wherein the dispensing portion is configured so that the dispensing port is inserted beneath the bursa premacularis of the eye.

9. The system of claim 1, further comprising an adhesive portion effective in affixing the drug delivery element to the retina of the eye.

10. The system of claim 9, wherein the adhesive portion circumscribes a portion of drug delivery element.

11. The system of claim 9, wherein the adhesive portion comprises a hydrogel material.

12. The system of claim 1, further comprising a biodegradable envelope substantially surrounding the drug delivery element and effective in enhancing the stability of the intravitreal implant when the implant is inserted into the vitreous of the eye.

13. The system of claim 12, wherein the envelope comprises a biodegradable polymeric component having a maximum expected life in the vitreous of less than about 24 hours.

14. The system of claim 12, wherein the envelope has a maximum external diameter of about 300 micrometers.

15. The system of claim 1, wherein the drug delivery element comprises a first biodegradable polymeric component, and wherein the drug delivery system further comprises an adhesive portion in contact with the drug delivery element and effective in affixing the drug delivery element in a substantially fixed position on the retina; and an envelope substantially surrounding the drug delivery element to form an intravitreal implant, the envelope comprising a biodegradable polymeric component different than the first biodegradable polymeric component and that degrades at a faster rate than the drug delivery element when the implant is placed in the vitreous of the eye.

16. The system of claim 15, wherein the dispensing port comprises a biodegradable polymeric component different than the first biodegradable polymeric component and the biodegradable polymeric component of the envelope, the dispensing port biodegradable polymeric component having diffusive resistance between the diffusive resistance of the first biodegradable polymeric component and the diffusive resistance of the biodegradable polymeric component of the envelope.

17. A method of manufacturing a therapeutic drug delivery system useful for placement into a posterior segment of an eye of an individual, comprising:
forming a polymeric material into a drug delivery element comprising a drug-containing reservoir portion and a drug dispensing portion extending from the reservoir portion, the drug dispensing portion having a dispensing port effective in providing directional delivery of the drug from the drug delivery element to a desired target location of an eye of an individual when the drug delivery element is fixedly positioned in the eye and is invisible to the individual.

18. The method of claim 17, wherein the polymeric material comprises a drug and the forming is effective in producing a matrix of the polymeric material and the drug, the drug being distributed substantially throughout the matrix.

19. The method of claim 17, wherein the forming comprises at least one step selected from the group consisting of extruding, injection molding, compression molding, tableting and machining, the polymeric material into the drug delivery element.

20. The method of claim 17, further comprising
forming an adhesive portion in contact with the drug delivery element; and
forming an polymeric envelope substantially surrounding the drug delivery element.

21. The method of claim 17, further comprising providing a second polymeric material on the dispensing port, the second polymeric material being removable from the dispensing port when the drug delivery element is located in the eye.

22. A method of treating an ocular condition of the posterior segment of an eye of an individual, comprising placing the drug delivery system of claim 1 into the posterior segment of the eye of the individual.

23. The method of claim 22, wherein the drug delivery system is placed near the macula of the eye so that the dispensing port delivers the drug to the macula.

24. The method of claim 22, further comprising inserting the dispensing port of the dispensing portion through the bursa premacularis of the eye.

25. The method of claim 22, further comprising affixing the drug delivery element to the retina.

26. The method of claim 25, wherein the drug delivery element comprises an adhesive portion, and the affixing comprises adhering the drug delivery element to the retina.
